(19) United States
(12) Patent Application Publication
(10) Pub. No.: US 2024/0366480 A1
(43) Pub. Date: Nov. 7, 2024

(54) Tinted Deodorant Composition

(71) Applicant: C'EST TOUT STUDIO, Holladay, UT (US)

(72) Inventor: Jozette Bateman, Holladay, UT (US)

(73) Assignee: C'EST TOUT STUDIO, Holladay, UT (US)

(21) Appl. No.: 18/647,613

(22) Filed: Apr. 26, 2024

Related U.S. Application Data
(60) Provisional application No. 63/463,092, filed on May 1, 2023.

(51) Int. Cl. A61K 8/18; A61K 8/02; A61Q 15/00

(52) U.S. Cl. CPC A61K 8/18; A61K 8/0229; A61Q 15/00; A61K 2800/43

(57) ABSTRACT

A deodorant composition may include a base. The deodorant composition may also include one of a deodorant and an antiperspirant distributed throughout the base. Additionally, the deodorant composition may include a tint mixture that mimics skin tone when distributed throughout the base.

### PRIORITY CLAIM

This application claims the priority and benefit of U.S. Provisional Patent Application No. 63/463,902 filed on May 1, 2023, which is incorporated herein by reference in its entirety, including but not limited to those portions that specifically appear hereinafter, the incorporation by reference being made with the following exception: In the event that any portion of the above-referenced applications are inconsistent with this application, this application supersedes said above referenced application.

### TECHNICAL FIELD

This disclosure relates generally to deodorants and antiperspirants, and more specifically to stick, roll-on, and soft solid deodorants and antiperspirants.

### BACKGROUND

Deodorants have been used since ancient times to mask unpleasant body odors. The first recorded use of deodorant dates back to ancient Egypt, where people used scented oils and perfumes to mask body odor. The Greeks and Romans also used perfumes and oils to mask body odor, and wealthy individuals used scented handkerchiefs to cover up smell from their sweat.

The first modern deodorant was developed in the late 19th century by an American inventor. This deodorant was made from a mixture of zinc oxide, starch, and fragrance. It was sold as a cream that was applied to the armpits with the fingers. It quickly became popular, and by the early 20th century, other companies were producing their own versions of deodorant.

Many deodorants also contain antiperspirant ingredients that reduce sweating as well as odor. In the 1940s, a new type of deodorant was developed that used aluminum chlorohydrate to reduce sweating. This type of deodorant, which is still used today, was more effective than earlier versions at reducing body odor. The popularity of deodorants has only continued to grow since then, with many different scents now available on the market. Today, deodorant has become an essential part of daily hygiene for many people.

Many different types of deodorants are available, including roll-ons, sprays, sticks, soft solids, and crystal deodorants. While deodorants are part of daily hygiene for many people, there are some problems associated with their use. For example, many deodorants can leave clearly visible residues, particularly white residues, at the application site which can be unsightly for users wearing clothing that exposes application sites. This issue is increasingly noticeable and problematic for users with skin tones that are darker than the white visible residues.

### SUMMARY

In some aspects, the techniques described herein relate to a deodorant composition, including: a base; one or more of a deodorant and an antiperspirant distributed throughout the base; and a tint mixture that mimics skin tone when distributed throughout the base.

In some aspects, the techniques described herein relate to a deodorant composition, including: a base including from 65 to 95 percent by mass of the composition; one of a deodorant and an antiperspirant including from 4 to 30 percent by mass of the composition distributed throughout the base; and a tint mixture that mimics skin tone when distributed throughout the base including from 0.05 to 10 percent by mass of the composition.

### BRIEF DESCRIPTION OF THE DRAWINGS

Non-limiting and non-exhaustive implementations of the present disclosure are described with reference to the following figures, wherein like or similar reference numerals refer to like or similar parts throughout the various views unless otherwise specified. Advantages of the present disclosure will become better understood with regard to the following description, and accompanying drawings:

FIG. 1 illustrates a skin-tone tinted deodorant composition.

FIG. 2 illustrates a tint mixture designed to mimic a relatively light skin tone when incorporated into a deodorant composition.

FIG. 3 illustrates a tint mixture designed to mimic a medium skin tone when incorporated into a deodorant composition.

FIG. 4 illustrates a tint mixture designed to mimic a relatively dark skin tone when incorporated into a deodorant composition.

### DETAILED DESCRIPTION

In the following description, for purposes of explanation and not limitation, specific techniques and embodiments are set forth, such as particular techniques and configurations, in order to provide a thorough understanding of the device disclosed herein. While the techniques and embodiments will primarily be described in context with the accompanying drawings, those skilled in the art will further appreciate that the techniques and embodiments may also be practiced in other similar devices.

Reference will now be made in detail to the exemplary embodiments, examples of which are illustrated in the accompanying drawings. Wherever possible, the same or similar reference numbers are used throughout the drawings to refer to the same or similar parts. It is further noted that elements disclosed with respect to particular embodiments are not restricted to only those embodiments in which they are described. For example, an element described in reference to one embodiment or figure, may be alternatively included in another embodiment or figure regardless of whether or not those elements are shown or described in another embodiment or figure. In other words, elements in the figures may be interchangeable between various embodiments disclosed herein, whether shown or not.

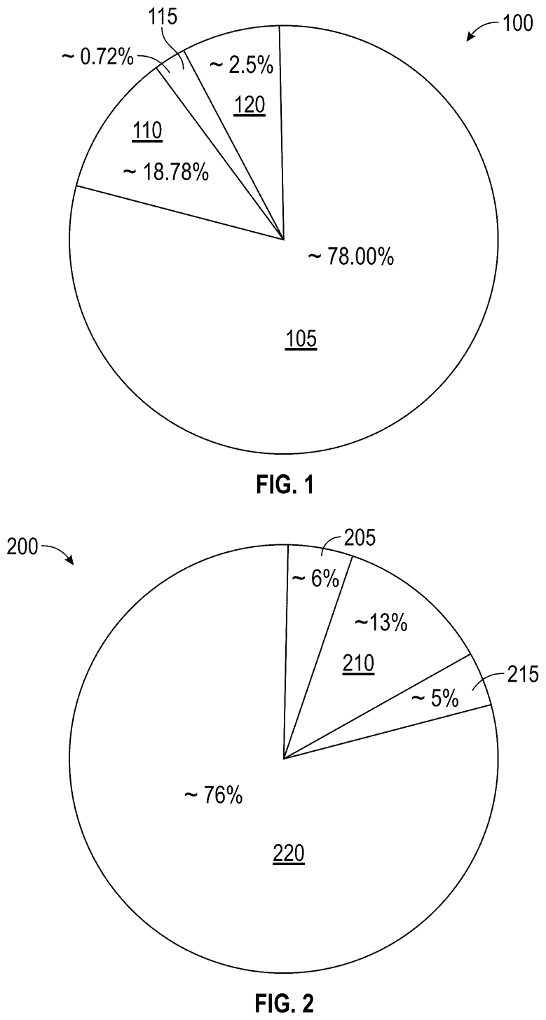

FIG. 1 illustrates a skin-tone tinted deodorant composition 100. Composition 100 may include a base 105, a deodorant 110, a tint mixture 115, and a fragrance 120. Each of deodorant 110, tint mixture 115, and fragrance 120 may be combined within and distributed throughout base 105 to create a homogenous mixture. Composition 100 may be provided as a roll-on or stick deodorant or other type of deodorant known in the art.

Base 105 may be formulated to give composition 100 various desirable characteristics. These characteristics may include ease of application, a silky texture, emulsification and delivery of specific ingredients, rapid drying, a powdery residual feel, stability and gelling of the deodorant composition, thickening of the composition, moisturization of the skin, absorption of excess moisture, and protection of the skin against allergens, bacteria, and moisture loss.

To achieve these characteristics, base 105 may comprise a variety of products such as cyclopentasiloxane, stearyl alcohol, c12-17 alkane, c14-22 alkane, hydrogenated castor oil, c10-18 triglycerides, peg-8 distearate, polyethylene, distarch phosphate, and other cosmetic ingredients known to a person having ordinary skill in the art. The specific composition of base 105 may be varied according to the desired properties of composition 100.

Deodorant 110 may include deodorizing agents, also referred to herein as deodorants, antiperspirant agents, also referred to herein as antiperspirants, agents that perform both deodorizing and perspiration-preventing functions, which may be referred to herein as either deodorants or antiperspirants, or any combination thereof. Examples of such agents may include aluminum zirconium tetrachlorohydrex GLY, aluminum chlorohydrate, aluminum zirconium tricholorohydrex glycine, aluminum chlorohydrate, aluminum hydroxybromide, cyclomethicone, triclosan, or other agents known to a person having ordinary skill in the art.

Deodorant 110 may be formulated to provide effective and long-lasting odor control, sweat reduction, non-toxicity, and other characteristics known to a person having ordinary skill in the art. The specific composition of the deodorant may vary depending on the desired level of deodorization, antiperspirant activity, toxicity risk, and other factors known to a person having ordinary skill in the art. The resulting product may thus provide an effective solution to body odor and/or sweat control.

Tint mixture 115 may be formulated to give composition 100 a specific color. For example, tint mixture 115 may be formulated to mimic any skin tone of human beings. Tint mixture 115 may comprise cosmetic colorants including organic colorants, inorganic colorants, water-soluble dies, natural materials such as carrot oil, beet extract, henna, and other cosmetic colorants known to a person having ordinary skill in the art.

In some embodiments, tint mixture 115 may include organic, inorganic, mineral, and other cosmetic colorants in a composition designed to mimic skin tones. For example, red, yellow, black, and white colorants may be combined to make tint mixture 115 mimic a desired skin tone. Tint mixture 115 may include cosmetic colorants approved for use in cosmetic products, for example, Color Index Number (“CI”) 77491 (or Red Iron Oxide), CI 77492 (or Yellow Iron Oxide), CI 77499 (or Black Iron Oxide), CI 77891 (or Titanium Dioxide), and other colorants. In the foregoing example, the color index numbers reference ingredient names as recognized by the International Nomenclature of Cosmetic Ingredients. Lists of specific colorants may be available through governing bodies, for example, the United States Food and Drug Administration.

As an example, and not by way of limitation, in some embodiments, colorants may include CI 77491 and 77499 for a red colorant, CI 77492 for a yellow colorant, CI 77499 for a black colorant, and CI 77891 for a white colorant. The specific colorants and relative quantities or amounts of the colorants in tint mixture 115 may vary depending on a desired skin tone as discussed in further detail below. The resulting tint mixture added to composition 100 may result in a skin-tone tinted deodorant composition that may exhibit an improved user experience due at least to the skin-tone-mimicking characteristics imparted by tint mixture 115 as discussed in further detail below.

Fragrance 120 may include a blend of essential oils, synthetic aroma chemicals, and other aromatic components known to a person having ordinary skill in the art to emit a desired scent. For example, fragrance 120 may comprise lavender, eucalyptus, peppermint, sandalwood, citrus, rose, jasmine, vanilla, musk, amber, and cedarwood. The components of fragrance 120 may be used individually or in combination with each other to create unique scent profiles for composition 100.

In one embodiment, composition 100 may include components in the following quantities: base 105 at approximately 78.00 percent by mass (“% m/m”), deodorant 110 at approximately 18.78% m/m, tint mixture 115 at approximately 0.72% m/m, and fragrance 120 at approximately 2.5% m/m as shown in FIG. 1. The relative quantities of the components of composition 100, however, may vary depending on the desired final characteristics of composition 100. For example, a user may desire a composition without fragrance, with a darker or lighter tint, with more or less deodorizing or perspiration-preventing capability, with more or less moisturizing capability, and so forth.

To achieve this flexibility in the design of composition 100, embodiments of composition 100 may include components adjustable between the following percent by mass ranges: base 105 from approximately 65 to 95% m/m, deodorant 110 from approximately 4 to 30% m/m, tint mixture 115 from approximately 0.05 to 10.00% m/m, and fragrance 120 from 0 to approximately 25% m/m.

Further, components may be adjustable within narrower ranges than those stated above. For example, composition 100 may include base 105 from approximately 65 to 85% m/m, from 70 to 95% m/m, and so forth. Similarly, composition 100 may include deodorant 110 from approximately 4 to 25% m/m, from 10 to 30% m/m, and so forth. Likewise, composition 100 may include tint mixture 115 from approximately 0.05 to 8% m/m, from 0.1 to 10.00% m/m, and so forth. Additionally, composition 100 may include fragrance 120 from 0 to approximately 20% m/m, from 1 to 25% m/m, and so forth.

Composition 100 may be applied topically to the underarm and other areas prone to sweating and odor. A user may enjoy an enhanced user experience due at least to the skin-tone-mimicking nature of composition 100. For example, a user may apply composition 100 on any area of exposed skin or on areas of skin that may be exposed at a later time without concern for the unsightliness of visible residue while still enjoying the hygienic benefits of composition 100. Composition 100 may be tinted to match an individual skin tone or color such that any left over residue will maintain the individual skin tone or color. Multiple tint mixtures are possible to mimic each person's different skin tone, whether the skin tone is darker, medium, or lighter. Skin tone may refer to an amount of pigmentation naturally present in the skin of a person where some people may have high pigmentation resulting in a dark skin tone, and some people may have low pigmentation resulting in a light skin tone, and anywhere in between for dark skin tones, medium skin tones, and light skin tones.

FIG. 2 illustrates a tint mixture 200 designed to mimic a relatively light skin tone when incorporated into composition 100. Tint mixture 200 may include red colorant 205, yellow colorant 210, black colorant 215, and white colorant 220. Tint mixture 200 is similar in implementation and description to the corresponding element shown in FIG. 1.

In one embodiment, tint mixture 200 may include components in the following quantities: red colorant 205 at approximately 6% m/m, yellow colorant 210 at approximately 14% m/m, black colorant 215 at approximately 5% m/m, and white colorant 220 at approximately 76% m/m as shown in FIG. 2. In this embodiment, when properly mixed with the other components of composition 100, tint mixture 200 is designed to cause composition 100 to mimic a relatively light skin tone. Thus, a user with a relatively light skin tone may topically apply composition 100 with this embodiment of tint mixture 200 to exposed areas of skin without concern for the unsightliness of visible residue while still enjoying the hygienic benefits of composition 100.

The relative quantities of the components of tint mixture 200, however, may vary depending on the desired final characteristics of composition 100. For example, a user may desire a composition with a darker or lighter tint. To achieve this flexibility in the design of tint mixture 200 and composition 100, embodiments of tint mixture 200 may include components adjustable between the following percent by mass ranges: red colorant 205 from approximately 0 to 70% m/m, yellow colorant 210 from approximately 0 to 70% m/m, black colorant 215 from approximately 0 to 80% m/m, and white colorant 220 from approximately 0 to 95% m/m.

Further, components may be adjustable within narrower ranges than those stated above. For example, tint mixture 200 may include red colorant 205 from approximately 0 to 65% m/m, from 5 to 70% m/m, and so forth. Similarly, tint mixture 200 may include yellow colorant 210 from approximately 0 to 65% m/m, from 5 to 70% m/m, and so forth. Likewise, tint mixture 200 may include black colorant 215 from approximately 0 to 75% m/m, from 5 to 80% m/m, and so forth. Additionally, tint mixture 200 may include white colorant 220 from approximately 0 to 90% m/m, from 5 to 95% m/m, and so forth.

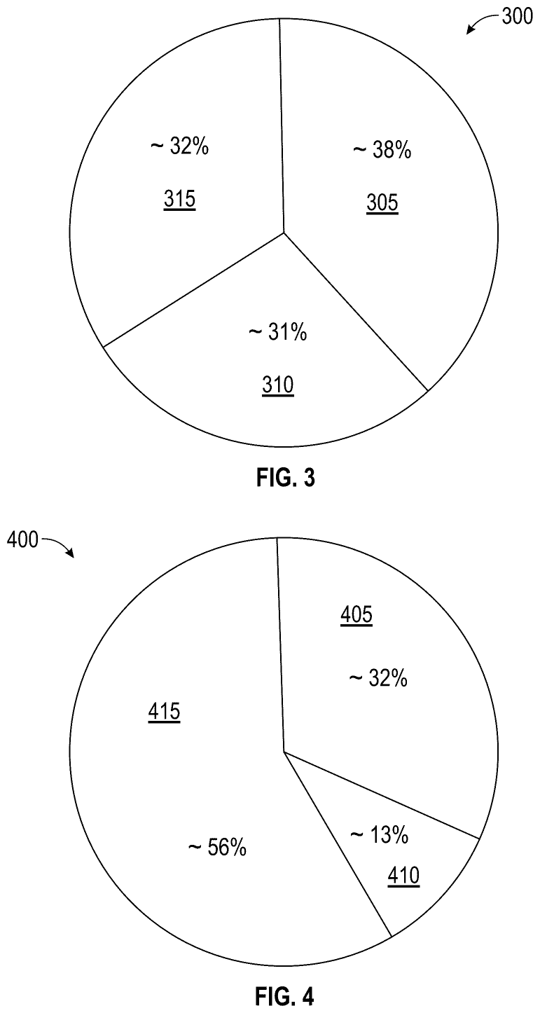

FIG. 3 illustrates a tint mixture 300 designed to mimic a medium skin tone when incorporated into composition 100. Tint mixture 300 may include red colorant 305, yellow colorant 310, and black colorant 315, each similar in implementation and description to the corresponding element shown in FIG. 2.

In one embodiment, tint mixture 300 may include components in the following quantities: red colorant 305 at approximately 38% m/m, yellow colorant 310 at approximately 31% m/m, and black colorant 315 at approximately 32% m/m, as shown in FIG. 3. In this embodiment, when properly mixed with the other components of composition 100, tint mixture 300 is designed to cause composition 100 to mimic a medium skin tone. Thus, a user with a medium skin tone may topically apply composition 100 with this embodiment of tint mixture 300 to exposed areas of skin without concern for the unsightliness of visible residue while still enjoying the hygienic benefits of composition 100.

The relative quantities of the components of tint mixture 300, however, may vary depending on the desired final characteristics of composition 100. For example, a user may desire a composition with a darker or lighter tint. To achieve this flexibility in the design of tint mixture 300 and composition 100, embodiments of tint mixture 300 may include components adjustable between the following percent by mass ranges: red colorant 305 from approximately 0 to 70% m/m, yellow colorant 310 from approximately 0 to 70% m/m, black colorant 315 from approximately 0 to 80% m/m, and a white colorant from approximately 0 to 95% m/m.

Further, components may be adjustable within narrower ranges than those stated above. For example, tint mixture 300 may include red colorant 305 from approximately 0 to 65% m/m, from 5 to 70% m/m, and so forth. Similarly, tint mixture 300 may include yellow colorant 310 from approximately 0 to 65% m/m, from 5 to 70% m/m, and so forth. Likewise, tint mixture 300 may include black colorant 315 from approximately 0 to 75% m/m, from 5 to 80% m/m, and so forth. Additionally, tint mixture 300 may include white colorant 320 from approximately 0 to 90% m/m, from 5 to 95% m/m, and so forth.

FIG. 4 illustrates a tint mixture 400 designed to mimic a relatively dark skin tone when incorporated into composition 100. Tint mixture 400 may include red colorant 405, yellow colorant 410, and black colorant 415, each similar in implementation and description to the corresponding elements shown in FIG. 2 and FIG. 3.

In one embodiment, tint mixture 400 may include components in the following quantities: red colorant 405 at approximately 32% m/m, yellow colorant 410 at approximately 13% m/m, and black colorant 415 at approximately 56% m/m, as shown in FIG. 4. In this embodiment, when properly mixed with the other components of composition 100, tint mixture 400 is designed to cause composition 100 to mimic a relatively dark skin tone. Thus, a user with a relatively dark skin tone may topically apply composition 100 with this embodiment of tint mixture 400 to exposed areas of skin without concern for the unsightliness of visible residue while still enjoying the hygienic benefits of composition 100.

The relative quantities of the components of tint mixture 400, however, may vary depending on the desired final characteristics of composition 100. For example, a user may desire a composition with a darker or lighter tint. To achieve this flexibility in the design of tint mixture 400 and composition 100, embodiments of tint mixture 400 may include components adjustable between the following percent by mass ranges: red colorant 405 from approximately 0 to 70% m/m, yellow colorant 410 from approximately 0 to 70% m/m, and black colorant 415 from approximately 0 to 80% m/m, and a white colorant from approximately 0 to 95% m/m.

Further, components may be adjustable within narrower ranges than those stated above. For example, tint mixture 400 may include red colorant 405 from approximately 0 to 65% m/m, from 5 to 70% m/m, and so forth. Similarly, tint mixture 400 may include yellow colorant 410 from approximately 0 to 65% m/m, from 5 to 70% m/m, and so forth. Likewise, tint mixture 400 may include black colorant 415 from approximately 0 to 75% m/m, from 5 to 80% m/m, and so forth. Additionally, tint mixture 400 may include white colorant 420 from approximately 0 to 90% m/m, from 5 to 95% m/m, and so forth.

Embodiments will be useful to users who desire the hygienic benefits of deodorants and/or antiperspirants while avoiding the problems of unsightliness caused by product residues in exposed areas of application. In particular, embodiments will be useful to users whose skin tone differs significantly from the residue colors associated with prior deodorant and antiperspirant products. It is also noted that specific compositions in FIGS. 2-4 are specific to particular skin tones and an infinite, or virtually infinite number of skin tone combinations are possible, which is appreciated by those of skill in the art. Specific or custom compositions may be formulated for the vast rainbow of different skin tones to serve people of all skin tones and are not limited to those embodiments shown in FIGS. 2-4.

Although the present disclosure has been described in considerable detail with reference to certain preferred versions thereof, other versions are possible. Therefore, the spirit and scope of the appended claims should not be limited to the description of the preferred versions contained therein.

What is claimed is:

1. A deodorant composition, comprising:
a base;
one or more of a deodorant and an antiperspirant distributed throughout the base; and
a tint mixture that mimics a skin tone when distributed throughout the base.

2. The deodorant composition of claim 1, further comprising a fragrance distributed throughout the base.

3. The deodorant composition of claim 1, wherein the tint mixture includes two or more of a yellow tint color, a red tint color, and a black tint color.

4. The deodorant composition of claim 3, wherein the tint mixture further includes a white tint color.

5. The deodorant composition of claim 3, wherein an amount of the two or more of the yellow tint color, the red tint color, and the black tint color in the tint mixture is selected for the deodorant composition to mimic a selected skin tone.

6. The deodorant composition of claim 3, wherein the tint mixture mimics a dark skin tone.

7. The deodorant composition of claim 3, wherein the tint mixture mimics a medium skin tone.

8. The deodorant composition of claim 3, wherein the tint mixture mimics a light skin tone.

9. The deodorant composition of claim 1, wherein the tint mixture includes a red colorant which comprises 6 to 38 percent of the tint mixture by mass, a yellow colorant which comprises 13 to 31 percent of the tint mixture by mass, and a black colorant which comprises 5 to 56 percent of the tint mixture by mass.

10. The deodorant composition of claim 9, wherein the base comprises 65 to 95 percent of mass of the deodorant composition.

11. A deodorant composition, comprising:
a base comprising from 65 to 95 percent by mass of the composition;
one or more of a deodorant and an antiperspirant comprising from 4 to 30 percent by mass of the composition distributed throughout the base; and
a tint mixture that mimics a skin tone when distributed throughout the base comprising from 0.05 to 10 percent by mass of the composition.

12. The deodorant composition of claim 11, further comprising a fragrance distributed throughout the base.

13. The deodorant composition of claim 11, wherein the tint mixture includes two or more of a yellow tint color, a red tint color, and a black tint color.

14. The deodorant composition of claim 13, wherein the tint mixture further includes a white tint color.

15. The deodorant composition of claim 13, wherein an amount of the two or more of the yellow tint color, the red tint color, and the black tint color in the tint mixture is selected for the deodorant composition to mimic a selected skin tone.

16. The deodorant composition of claim 13, wherein the tint mixture mimics a dark skin tone.

17. The deodorant composition of claim 13, wherein the tint mixture mimics a medium skin tone.

18. The deodorant composition of claim 13, wherein the tint mixture mimics a light skin tone.

19. The deodorant composition of claim 11, wherein the tint mixture includes a red colorant which comprises 6 to 38 percent of the tint mixture by mass, a yellow colorant which comprises 13 to 31 percent of the tint mixture by mass, and a black colorant which comprises 5 to 56 percent of the tint mixture by mass.

20. The deodorant composition of claim 11, provided as one of a roll-on or stick deodorant.
